(19) United States
(12) Patent Application Publication
(10) Pub. No.: US 2006/0161190 A1
(43) Pub. Date: Jul. 20, 2006

(54) Disposable laparoscopic instrument

(76) Inventors: Donald L. Gadberry, San Clemente, CA (US); Gary M. Johnson, Mission Viejo, CA (US); Jeremy J. Albrecht, Ladera Ranch, CA (US); David Okihisa, Irvine, CA (US); Said S. Hilal, Coto de Caza, CA (US); Arkadiusz A. Strokosz, Dana Point, CA (US)

Correspondence Address: APPLIED MEDICAL RESOUCES CORPORATION, 22872 Avenida Empresa, Rancho Santa Margarita, CA, 92688, US

(21) Appl. No.: 11/334,027

(22) Filed: Jan. 18, 2006

Related U.S. Application Data
(60) Provisional application No. 60/725,234, filed on Oct. 11, 2005.
(60) Provisional application No. 60/665,069, filed on Mar. 24, 2005.
(60) Provisional application No. 60/645,319, filed on Jan. 19, 2005.

(51) Int. Cl. A61B 17/32; A61B 17/28

(52) U.S. Cl. 606/174; 606/205

(57) ABSTRACT

The invention is directed to a surgical instrument including a handle assembly and a shaft assembly. The handle assembly further includes a fixed handle and a pivoting handle. The shaft assembly extends from the handle assembly and further includes an outer tube and an inner actuation rod that slides coaxially with the outer tube. The shaft assembly includes a rotatable knob to provide 360° rotation. The actuation rod has a proximal end and a distal end; the proximal end has a ball end that couples with the pivoting handle to form a rotatable ball-and-socket joint. The outer tube is formed of plastic and fits over the actuation rod to function as an electrical insulator.

This application fully incorporates by reference and claims priority to: provisional application, Ser. No. 60/725,234, filed by Applicants on Oct. 11, 2005, entitled “Disposable Laparoscopic Instrument;” provisional application, Ser. No. 60/665,069, filed by Applicants on Mar. 24, 2005, entitled “Disposable Laparoscopic Instrument;” and provisional application, Ser. No. 60/645,319, filed by Applicants on Jan. 19, 2005, entitled “Disposable Laparoscopic Instrument.”

### BACKGROUND OF THE INVENTION

This invention generally relates to surgical instruments and, more specifically, to a disposable laparoscopic instrument having jaw members that pivot in response to the opening and closing of a handle member, where movement of the handles is translated through a shaft member to open and close the jaw members and to facilitate access to distant operative sites.

It is often desirable to cut tissue, occlude vessels or perform some other surgical procedure at a distant operative site. Under these circumstances, a surgical instrument having an elongate shaft assembly is typically required. Such a shaft assembly might have an operative mechanism, such as a clamp or scissors, at its distal end, and a handle assembly at its proximal end for operating the instrument through the shaft assembly.

In the past, each instrument was formed as a single structure with its own handle assembly, shaft assembly, and associated operative mechanism. For example, many of these instruments provide an intricate construction in which a linkage mechanism for opening and closing the jaw members requires numerous moving parts, while a sliding arrangement is provided between two extended rod members that activate the linkage mechanism in response to movement of the handle members. Unfortunately, the complexity of the mechanics involved in these instruments has not changed much and has made it difficult to achieve adequate sterilization for reuse. In one conventional clamp apparatus, for example, a cable is permanently secured in the handle assembly and the shaft may be detachable from the handle assembly. The typical means for joining the handle assembly and the shaft includes an externally threaded connector on the shaft that mates with an internally threaded barrel of the handle assembly. A drawback of this apparatus is the difficulty of connecting together the shaft and the handle assembly, as well as the complexity of internal components required to achieve a functional two-piece device. Moreover, as these instruments have continued to be manufactured as two-piece structures, the problems relating to sterilization, access and overall cost have remained.

### SUMMARY OF THE INVENTION

The disposable laparoscopic instrument of the invention overcomes many of the disadvantages of the prior art and provides an instrument that is easy to manufacture and use. In one aspect, the invention is directed to a surgical instrument including a handle assembly and a shaft assembly. The handle assembly further includes a fixed handle and a pivoting handle. The shaft assembly extends from the handle assembly and further includes an outer tube and an inner actuation rod that slides coaxially with the outer tube. The shaft assembly may be a 5 mm diameter shaft having an accessible knob providing 360° rotation. The outer tube is coupled to the rotatable knob. The actuation rod has a proximal end and a distal end; the proximal end has a ball end that couples with the pivoting handle to form a rotatable ball-and-socket joint. The ball end of the actuation rod fits into a groove in the pivoting handle to permit the actuation rod of the shaft assembly to self-align as the pivoting handle moves. Once assembled, the fixed handle encloses the pivoting handle to prevent the ball end from pulling out of the groove under load.

The distal end of the actuation rod is coupled to an operative mechanism. In one embodiment, the operative mechanism includes a clamp having an inner blade and an 1o outer blade. It is appreciated that the operative mechanism may be provided with a variety of different operative mechanisms having different body functions, thereby expanding the capabilities of the surgical instrument. For example, the operative mechanism may include a clamp, a pair of scissors, or a balloon device. It is further appreciated that the operative mechanism may include any device that utilizes the actuation rod to move between a first state and a second state. The inner blade and outer blade may be coupled to an insert having an aperture to accept a pin, such as a rivet pin, dowel pin or screw, that allows the operative mechanism to pivot during opening and closing. The insert may be coupled or press-fit to a distal-end portion of the outer tube. The insert and outer tube can be sized and configured so as to permit greater opening angles for the blades. The insert may be formed of a plastic or metallic material. The insert may also be coupled to the outer tube by adhesive or other coupling means. In one aspect, the insert may include a distal clevis and a proximal clevis. The proximal clevis extends over the proximal ends of the blades and sandwiches the proximal ends of the blades between the actuation rod and the walls of the proximal clevis to contain the proximal ends of the blades from moving away from the actuation rod and out of their drive slots within the actuation slots.

In another aspect of the invention, the handle assembly may further include a connecting post to provide for cauterization of tissue during a procedure. More specifically, the connecting post includes a spring and a connector and may be formed in the fixed handle so as to extend from the top either at an angle or perpendicular to the actuation rod. The connecting post may be contained in the fixed handle by means of a loose fit to allow it to freely rotate 360°, or by force fit, adhesive, threads or other means. With this aspect, the spring extends from the connecting post to make contact with the actuation rod as it rotates and/or moves axially to provide electrical contact. The spring may be a round helical compression spring or a flat wire cantilever spring. As the electrical charge is applied through the connecting post to the actuation rod and then to the operative mechanism such as the blades, the operator is protected from electrical shock as both the handle assembly and the outer tube of the shaft assembly are formed from plastic material and thus serve as an electrical insulation barrier. The outer tube is formed of a thermoplastic or a thermoset plastic. The outer tube fits over the actuation rod to function both as a structural member and as an insulator to prevent electrical shock. It is appreciated that the outer diameter of the actuation rod fits closely to the inner surface of the outer tube so as to support it. With this aspect, both the actuation rod and the blades are formed of corrosion resistant steel, but it is appreciated that the actuation rod and the operative mechanism can be formed of any electrically conducting and corrosion resistant material.

In another aspect, the knob has internal key couplings for rotating the outer tube of the shaft assembly. Specifically, the knob may further include a hub, an alignment hole, and a retaining pin or other similar retention means for coupling the knob to the handle assembly to allow 360° rotation. The handle assembly may include a circumferential groove where a retaining pin of the knob is to be placed, inserted or extended to allow rotation. The knob may further include a spring washer between the distal end of the handle assembly and the proximal end of the hub to take up the linear play due to tolerance variations. The hub may be formed of a plastic material and may be overmolded onto the outer tube to provide secure coupling. The hub may also be coupled to the outer tube with an adhesive or other coupling means. The rotatable knob may also have a plurality of ribs on its inside diameter, providing a thin-walled knob while maintaining stability and reducing rotating friction with the handle assembly and the shaft assembly. The openings between the ribs allow for a generous draft angle, resulting in improved part ejecting during the molding process.

In another aspect of the invention, the fixed handle is provided with a first snap-in ring insert to fit a user's hand size and the pivoting handle is provided with a second snap-in ring insert to fit a user's hand size. A range of snap-in ring sizes for both the fixed handle and the pivoting handle may be provided the ring inserts are to be included with each handle assembly of the surgical instrument so a user can select, for example, a variety of sizes. The ring inserts are to be formed of a softer material than the handle material to provide comfort to the user. In addition, the ring inserts can be made of different colors for identification of various sizes. It is appreciated that the handle assembly of the surgical instrument can be used with or without the ring inserts.

In another aspect of the invention, a surgical instrument includes an elongate tube extending along an axis that includes an actuation rod that is coaxially slidable within the elongate tube. The surgical instrument also includes a first tip having a first pin formed on a proximal end surface of the first tip and a second tip having a second pin formed on a proximal end surface of the second tip. The second tip is pivotally coupled to the first tip at a common pivot pin that is operably connected to the elongate tube to open and close the tips in response to movement of the actuation rod. The actuation rod has at least one slot that accepts the pins of the first and second tips. The slot has camming surfaces for the pins to slide within the slot, and the proximal ends of the tips extend minimally outside the diameter of the elongate tube during actuation of the tips. In one aspect, the proximal ends of the tips do not extend outside the inner diameter of the elongate tube during actuation of the tips. In another aspect, the actuation rod includes a tongue portion at its distal end with two slots transverse to one another on opposing sides of the tongue. In another embodiment, the slots on the tongue portion are curved. In another embodiment, the slots on the tongue may be either open-end slots or closed-end slots. In another embodiment, the pins are formed on the proximal end surfaces of the tips by press fitting, threading, welding or bonding.

These and other features and advantages of the invention will become more apparent with a discussion of embodiments of the invention and reference to the associated drawings.

### DESCRIPTION OF THE DRAWINGS

FIG. 1 is a side view of one embodiment of the invention including a handle assembly, a shaft assembly and an operative mechanism;

FIGS. 2A-2C illustrate cross-sectional views of the connections between the handle assembly and the shaft assembly;

FIGS. 3A-3C illustrate a rear perspective view of the fixed handle, a front perspective view of the fixed handle, and a top perspective view of the pivoting handle, respectively;

FIGS. 4A and 4B illustrate a top view and a side view of an operative mechanism of the invention including an insert and a pair of blades, FIG. 4C illustrates a perspective view of the insert of the operative mechanism of FIGS. 4A and 4B, and FIG. 4D illustrates a cross-sectional view of an insert having a distal clevis and a proximal clevis in accordance with another aspect of the invention;

FIGS. 5A and 5B illustrate perspective views of the operative mechanism of the invention without the outer tube and with the outer tube of the shaft assembly, respectively;

FIGS. 5C-5D illustrate perspective and cross-sectional views of an insert for receiving a screw in accordance with another aspect of the invention;

FIG. 5E illustrates a side view of the device of FIG. 5D;

FIGS. 6A-6C illustrate perspective and side views of a handle assembly in accordance with another aspect of the invention including a connecting post to provide for cauterization of tissue during a procedure;

FIG. 7 illustrates a proximal view of a rotatable knob in accordance with another aspect of the invention having a plurality of ribs providing a thin-walled knob while maintaining stability and reducing friction with the handle assembly and the shaft assembly;

FIGS. 8A-8C illustrate the handle assembly of the invention providing a variety of sizes of snap-in ring inserts to fit a user's hand size;

FIGS. 9A-9D illustrate cross-sectional and side views of a fixed handle being formed of two pieces in accordance with another aspect of the invention;

FIGS. 10A-10C illustrate a perspective view of a laparoscopic surgical instrument of the invention, a perspective view of a blade or tip of the operative mechanism of the invention, and a side view of FIG. 10A, respectively;

FIG. 11 illustrates a perspective view of an assembled surgical instrument of the invention having mobile tips and an actuation rod;

FIGS. 12A and 12B illustrate a perspective view and a side view of an actuation rod having a slot with an open end in accordance with an aspect of the invention; and

FIGS. 12C and 12D illustrate a perspective view and a side view of an actuation rod having a curved slot in accordance with another aspect of the invention.

### DESCRIPTION OF THE INVENTION

The invention and its various embodiments can now be better understood with the following detailed description wherein illustrated embodiments are described. It is to be expressly understood that the illustrated embodiments are set forth as examples and not by way of limitations on the invention.

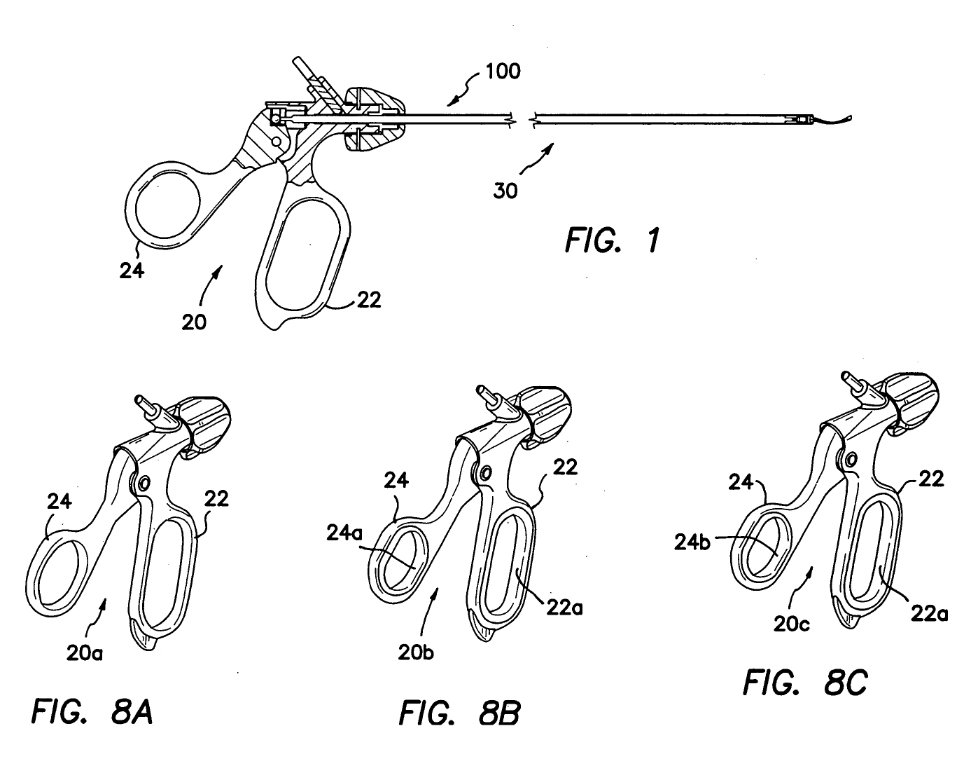

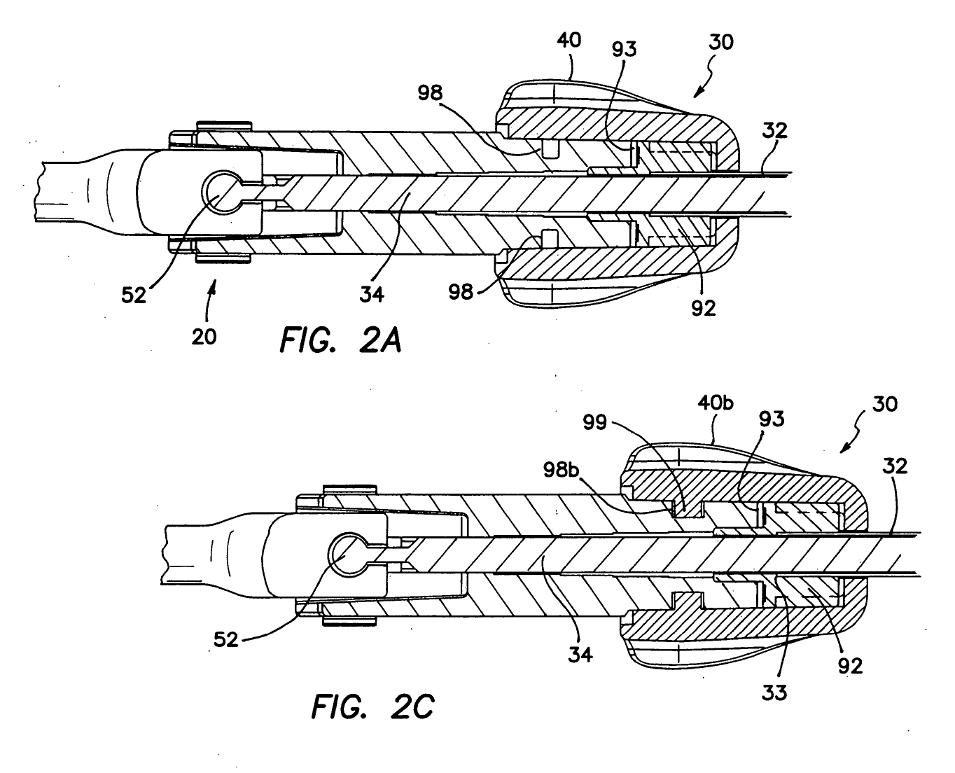

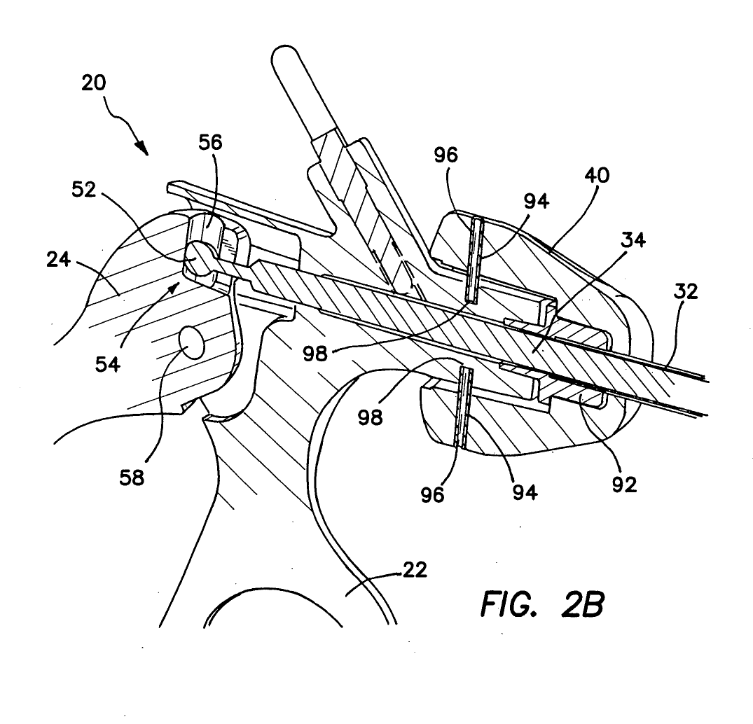

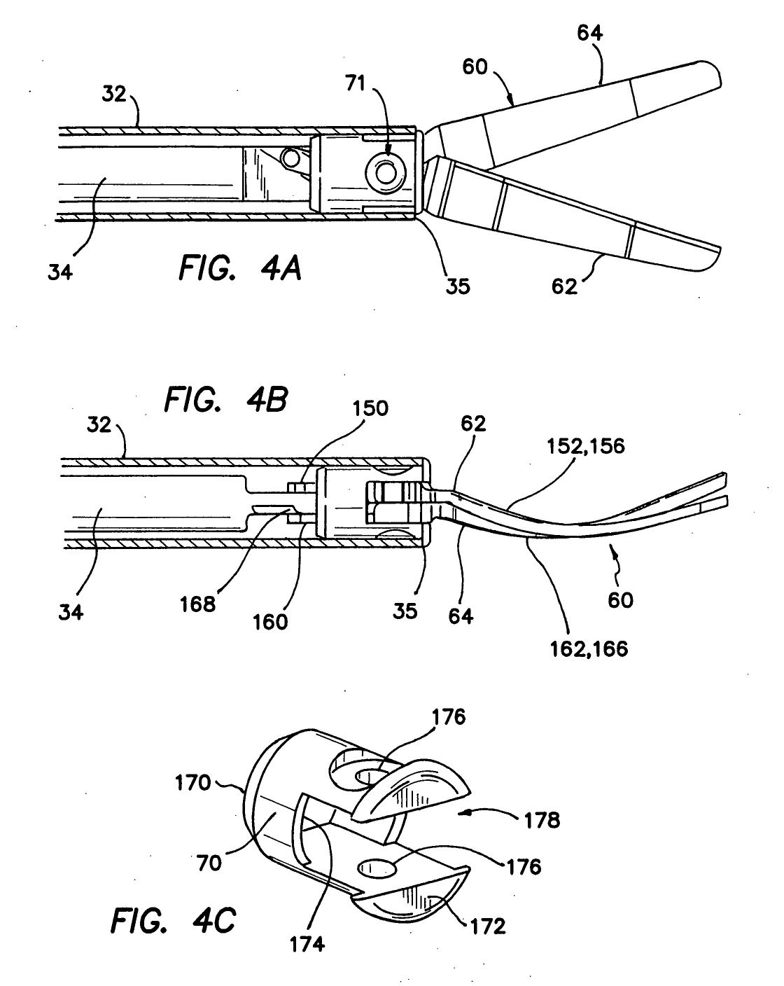

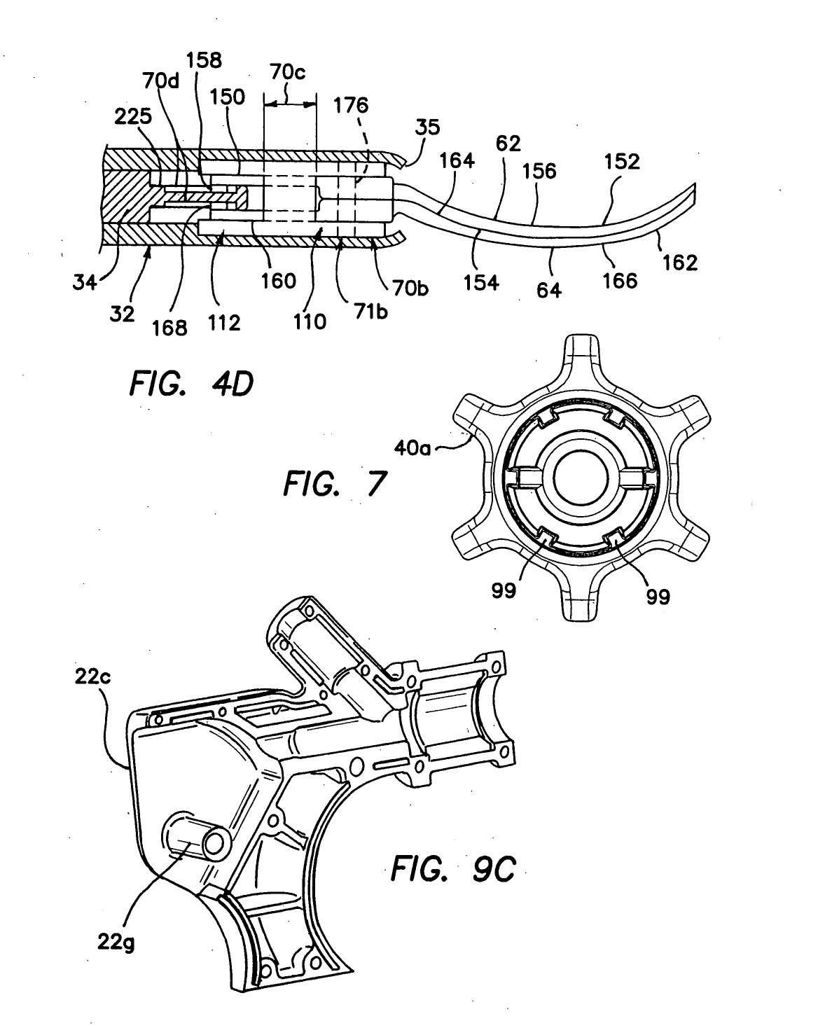

A first embodiment of a surgical instrument is illustrated in FIG. 1 and designated by the reference numeral 100. The surgical apparatus 100 includes a handle assembly 20 and a shaft assembly 30. The handle assembly 20 further includes a fixed handle 22 and a pivoting handle 24. Referring to FIGS. 2A and 2B, there are shown cross-sectional views of the connections between the handle assembly 20 and the shaft assembly 30. In particular, the shaft assembly 30 extends distally from the handle assembly 20 and further includes an outer tube 32 and an inner actuation rod 34 that slides coaxially within the outer tube 32. The shaft assembly 30 may be a 5 mm diameter shaft having an accessible knob 40 providing 360° rotation as further described below. The outer tube 32 is an elongate tube of any cross-sectional shape that extends along an axis and includes a proximal end 33 (see FIG. 2C) and a distal end 35 (see FIGS. 4A and 4B). The outer tube 32 is coupled to a rotatable knob 40 as further described below.

The actuation rod 34 includes a proximal end and a distal end. The proximal end of the actuation rod extends proximally from the proximal end 33 of the outer tube 32 and the distal end of the actuation rod is positioned within a distal-end region of the outer tube. The proximal end of the actuation rod has a ball end 52 that couples with the pivoting handle 24 to form a rotatable ball-and-socket joint. The ball end 52 of the actuation rod 34 fits into a groove 56 in the pivoting handle 24 to permit the actuation rod 34 of the shaft assembly 30 to self-align as the pivoting handle 24 moves. The outer tube 32 can be formed of a plastic material and the actuation rod 34 can be formed of a plastic or metallic material, depending on the application.

In contrast to the invention, surgical instruments of the prior art are typically formed so as to have a metal outer tube and a metal actuation rod. More specifically, the actuation rod is typically a small metal round rod or a flat stamped rod extending through the lumen of the metal outer tube between the handle assembly and the jaw members. With the prior art instruments, the metal outer tube provides flexural strength to the shaft assembly as the actuation rod moves back and forth inside the shaft assembly to operate the jaw members. The metal outer tube is then covered with one or more layers of plastic shrink tubing that acts as an electrical insulator between the shaft assembly and the patient and/or user. Accordingly, these prior art instruments require at least three components: an actuation rod; a metal outer tube to provide flexural strength; and a plastic shrink tubing to provide electrical insulation.

In comparison, the invention discloses an outer tube 32 that is formed of a plastic material and an inner actuation rod 34 that can be formed of plastic or metal, depending on the application. The actuation rod 34 can be a round or any other cross-sectional shape well known in the art, stamped or formed member that provides flexural strength to the shaft assembly 30. Because the outer tube 32 is formed of plastic, it is electrically nonconductive and does not require further insulating outer tubing. It should be noted that the plastic outer tube 32 provides both sufficient flexural and compression strength, yet it provides greater electrical insulative properties than a thin walled shrink tubing. The plastic outer tube 32 can be formed, for example, by extrusion. It is further appreciated that the process of assembling the plastic outer tube 32 is also simpler than that of forming the shrink tubing over the metal outer tube, and the costs involved with the process of the invention are also much less than those of the prior art. From the material supply standpoint, a plastic outer tube is also more timely available, since it can be extruded in-house, and any secondary operations, such as hole drillings through the proximal end for the handle assembly, cutting to precision length, insert molding a component onto the tube, and counterboring the distal end to provide an internal cavity and shoulder for the jaw members, can also be done in-house. The design of the invention thus facilitates and simplifies assembly.

Referring back to FIG. 2B, the ball end 52 can be formed from a plastic material at the proximal end of the actuation rod 34, and the actuation rod may include barbs or have an enlarged outer diameter. The plastic ball end 52 can then be force-fit onto the actuation rod 34 and fitted into the groove 56 to form the ball-and-socket joint or coupling. The ball end 52 can also be formed in a plurality of pieces, for example, two halves that are then joined together by any coupling means including a snap-fit connection, press-fit posts, or with adhesive. The actuation rod 34 can also be machined to accept the pieces of the ball end 52, and can be designed to provide s strength when the rod is experiencing a load. In one example, the ball end 52 may include a male half and a female half and the actuation rod 34 may be configured to accept the halves accordingly. Other benefits of having the ball end 52 made of plastic include lubricity of the plastic ball joint, ease of rotation, less likelihood of torsional lockup, and reduced stress on the pivot pin 58.

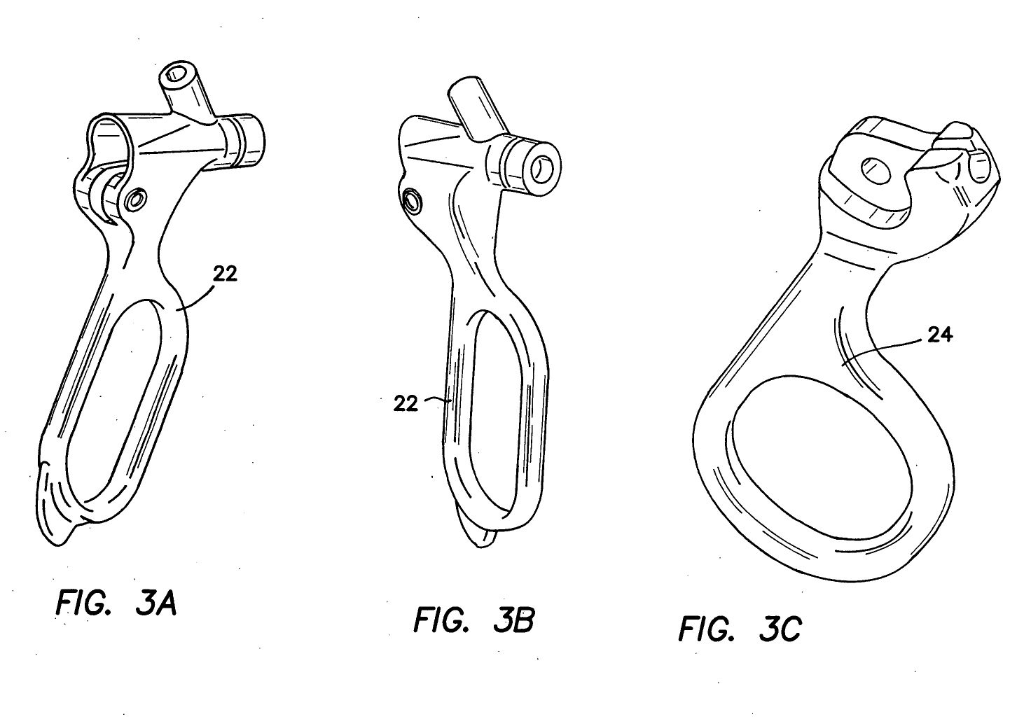

Once assembled, the fixed handle 22 encloses the pivoting handle 24 to prevent the ball end 52 from pulling out of the groove 56 under load. FIGS. 3A-3C illustrate a rear perspective view of the fixed handle 22, a front perspective view of the fixed handle 22, and a top perspective view of the pivoting handle 24, respectively. During assembly, the fixed handle 22 may be slid over the proximal end of the actuation rod 34 so as to expose the ball end 52, which may then be placed into groove 56 of the pivoting handle 24, and a pivot pin 58 (see FIG. 2B) may then be inserted or placed for retaining the fixed and pivoting handles 22, 24. The handle 24 pivots about the pivot pin 58 to move in relation to the fixed handle 22.

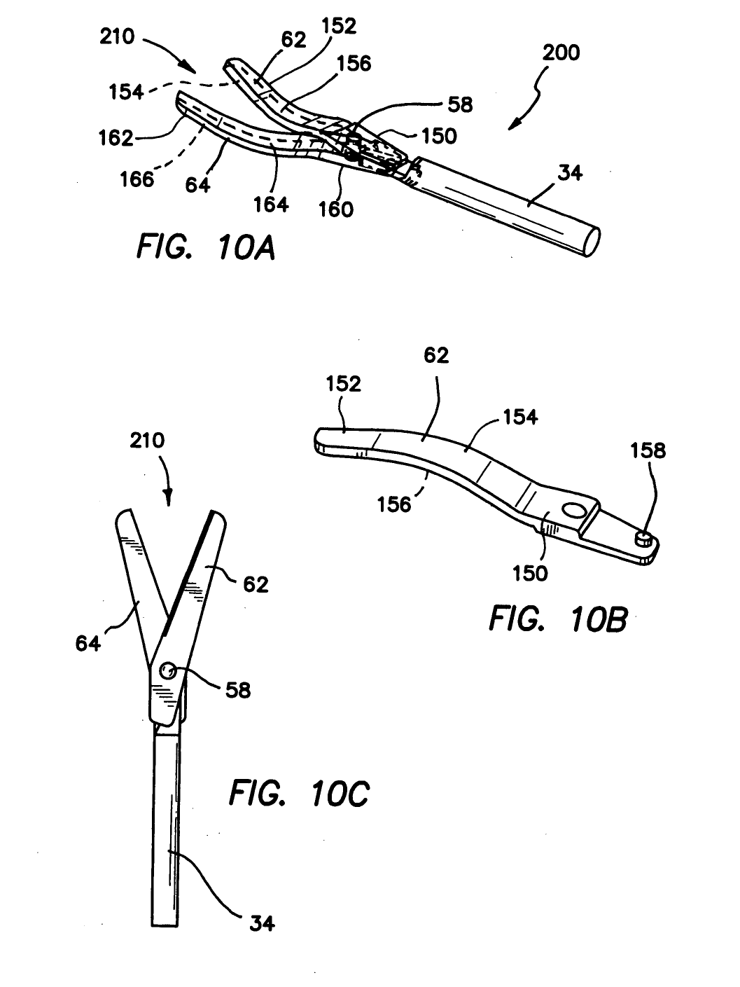

Referring to FIGS. 4A and 4B, the distal end of the actuation rod 34 is coupled to an operative mechanism 60. In one embodiment, the operative mechanism 60 includes a clamp or scissors having a first blade or tip 62 (inner blade) and a second blade or tip 64 (outer blade). The first tip 62 includes a proximal-end portion 150, a distal-end portion 152, an interfacing side 154 and an opposed side 156, with a first pin 158 protruding from the interfacing side of the proximal-end portion proximate a proximal end of the first tip (see FIGS. 10A and 10B). The second tip 64 includes a proximal-end portion 160, a distal-end portion 162, an interfacing side 164 and an opposed side 166, with a second pin 168 protruding from the interfacing side of the proximal-end portion proximate a proximal end of the second tip. The interfacing side 154 of the first tip 62 and the interfacing side 164 of the second tip 64 interface with each other. It is appreciated that the operative mechanism 60 can be provided with a variety of different operative mechanisms having different functions, thereby expanding the capabilities of the surgical apparatus 100. For example, the operative mechanism 60 can include a clamp, a pair of scissors, or a balloon device. It is further appreciated that the operative mechanism 60 can include any device that utilizes the actuation rod 34 to move between a first state and a second state.

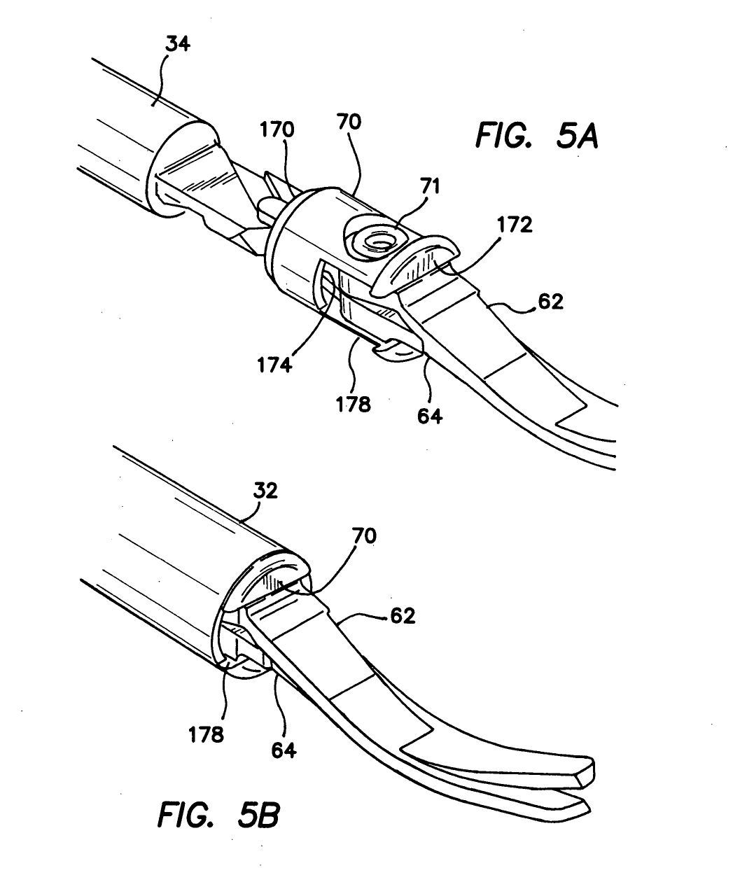

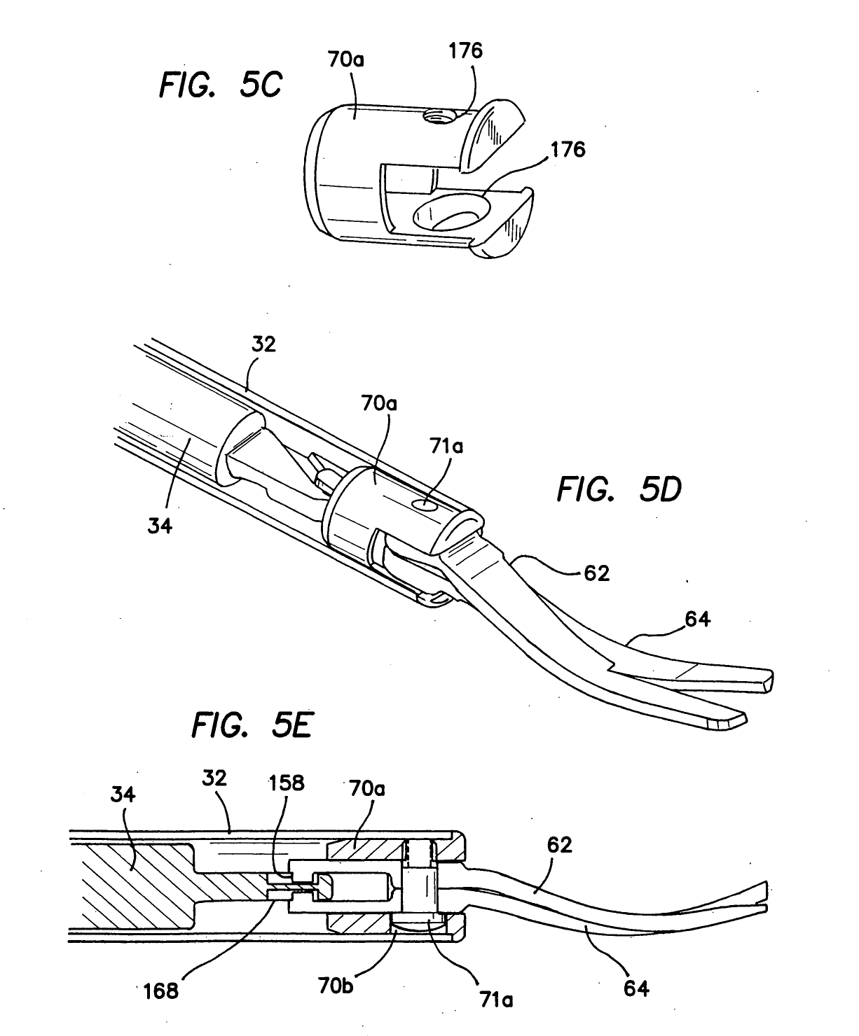

Referring to FIGS. 4C, 5A and 5B, the first tip 64 and second tip 62 are coupled to an insert 70. The insert 70 is a substantially cylindrical insert having a proximal end 170 and a distal end 172 with a lumen 174 therebetween. The insert 70 includes a radial hole or aperture 176 that is substantially perpendicular to an axis of the insert. The aperture 176 is substantially radial and is adapted to accept a rivet pin 71 or dowel pin that allows the operative mechanism 60 to pivot during opening and closing. The insert 70 includes a clevis 178 extending longitudinally in a proximal direction from its distal end 172. The clevis 178 is substantially parallel to the axis of the insert 70 and substantially perpendicular to the aperture 176 of the insert. The insert 70 may be coupled or press-fit to the outer tube 32. The first and second tips 62, 64 are retained within the clevis 178 of the insert 70 by the rivet pin 71. More particularly, at least a portion of the proximal-end portion 150 of the first tip 62 and the proximal-end portion 160 of the second tip 64 are positioned within the lumen 174 of the insert 70. The insert 70 can then be inserted into a distal end 35 of the outer tube 32 and retained therein by force-fit, adhesive, or crimping the end of the outer tube 32 over the end of the insert 70.

In another aspect, as illustrated in FIG. 4D, an insert 70b can have a distal clevis 110 and a proximal clevis 112. The distal clevis 110 and the proximal clevis 112 are substantially rotatably aligned with each other about the axis of the insert, substantially parallel to the axis of the insert, and substantially perpendicular to the aperture of the insert. With this aspect, the distal clevis 110 operates to hold the blades 62, 64 in place, a center portion 70c of the insert 70b provides a mechanical stop for blade travel (i.e., surfaces of the through hole or lumen 174 control the limits of blade travel in full open position), and the proximal clevis 112 extends over the proximal ends of the blades 62, 64 to contain the proximal ends of the blades from moving away from the actuation rod 34 and out of their drive slots 70d within the actuation rod. More particularly, the proximal portions 150, 160 of the blade are sandwiched between the tongue portion 225 of the actuation rod 34 and the walls of the proximal clevis portion of the insert 70b in order to substantially prevent pins at the proximal portions of the blades 62, 64 from becoming dislodged from the slots, as will be described below in greater detail. The opening in the proximal clevis 112 portion of the insert 70b permits the proximal portion of the blades 62, 64 to move further outwardly with the blades in the open position. The walls of the proximal clevis 112 in the insert 70b provide a cantilever force for the proximal portions of the blades 62, 64, which provides more efficient use of the blades.

The insert 70 can be formed in a plurality of pieces, e.g., two halves, by a casting, molding or other processes, in either a plastic or metallic material. A benefit of forming the insert 70 in multiple pieces is it is suitable for high production, easier to assemble, and less costly than a machined part. It is further appreciated that the outer tube 32 can have a counterbore at the distal end 35 to control the insertion depth of the insert 70, 70b and prevent the insert from being pulled further proximally into the outer tube. Alternatively, the outer tube 32 can be crimped or heat staked to retain the insert 70, 70b in the tube when the actuation rod 34 applies scissor-opening force. The insert 70, 70b and the outer tube 32 can also be sized and configured so as to permit greater opening angles for the blades 62, 64.

Referring to FIGS. 5C-5E, this aspect of the invention uses an insert 70a having screw threads to accept a screw 71a. In comparison to the rivet pin 71 of FIGS. 5A and 5B, the screw 71a provides a means for adjusting the fit of the blades 62, 64, which in turn adjusts the tension between the cutting surfaces of the blades 62, 64, whereas use of a rivet pin allows the blade tensioning to be consistently set during assembly, which provides consistency between surgical instruments 100. The hole or aperture 176 through the insert 70a permits the screw 71a to have a thicker head, as space is limited. It is appreciated that another configuration could employ a counterbore for the head of the screw 71a instead of the hole. The insert 70, 70a can be formed of, for example, a plastic or metallic material, depending on the application. The insert 70, 70a can also be coupled to the outer tube 32 by adhesive or other coupling means.

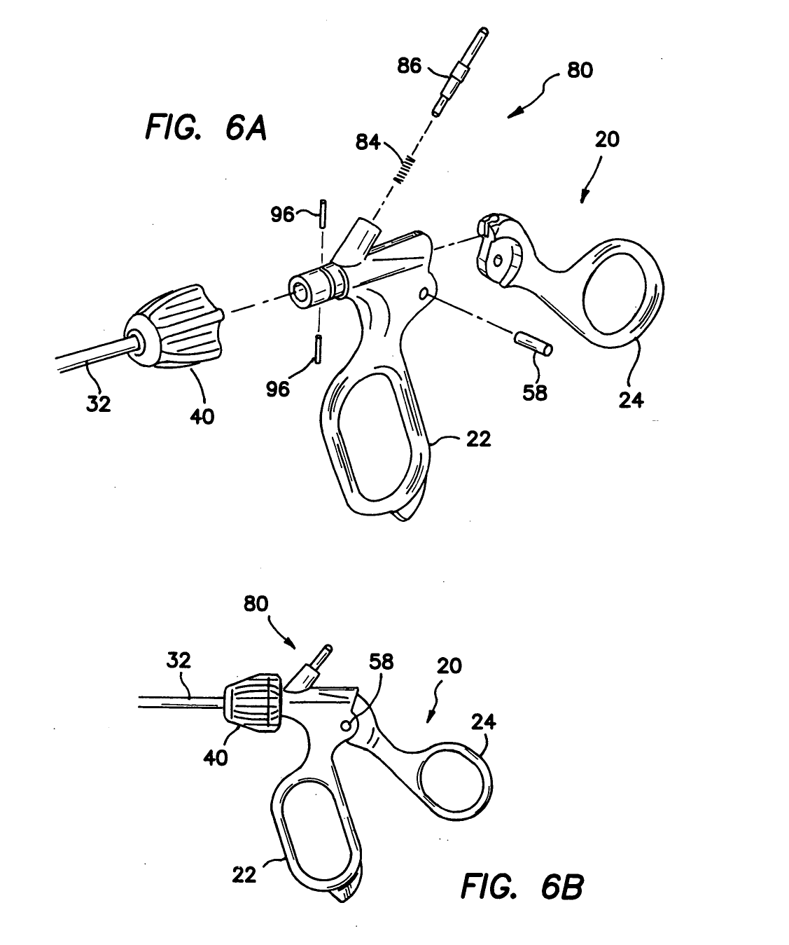

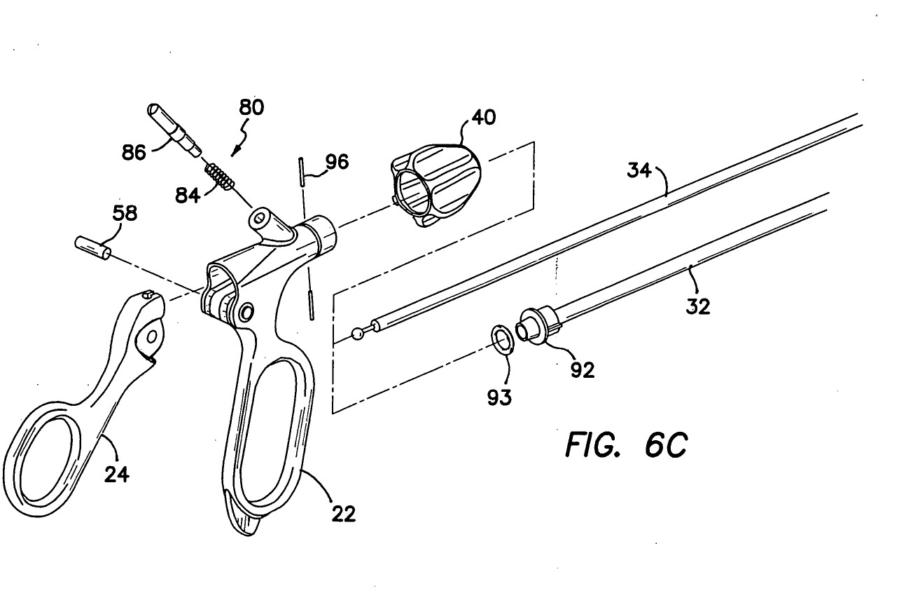

In another aspect of the invention, the handle assembly 20 may further include a connecting post 80 as illustrated in FIGS. 6A-6C to provide for cauterization of tissue during a procedure. More specifically, the connecting post 80 is formed in the fixed handle 22 so as to extend from the top either at an angle or perpendicular to the top. The connecting post 80 may be either freely contained in the fixed handle 22 or secured by force fit, adhesive, threads or other means, and may include a spring 84 and a connector 86. With this aspect of the invention, the spring 84 extends from the connecting post 80 into contact with the actuation rod 34 to provide electrical contact as the actuation rod rotates and/or moves axially. The spring 84 may be a round helical compression spring or a flat wire cantilever spring. As the electrical charge is applied to the actuation rod 34 and then to the operative mechanism 60, such as the blades 62 and 64, the operator is protected from electrical shock as both the handle assembly 20 and the outer tube 32 of the shaft assembly 30 are formed from plastic material and thus serve as an electrical insulation barrier. In particular, the outer tube 32 is formed of a thermoplastic or a thermoset plastic that fits over the actuation rod 34 and functions as an insulator to prevent electrical shock with the operator and/or patient. In one aspect, the outer tube 32 may also function as a structural member. It is appreciated that the outer diameter of the actuation rod 34 may be formed to fit closely to the inner diameter of the outer tube 32 so as to support it. With this aspect, the actuation rod 34 and the blades 62, 64 are formed of corrosion resistant steel, but it is appreciated that the actuation rod 34 and the operative mechanism 60 can be formed of any electrically conductive and corrosion resistant material.

Referring back to FIGS. 2A and 2B, the rotatable knob 40 is coupled to the exterior surface of the elongate tube 32 proximate the proximal end of the elongate tube and coupled to the handle assembly 20. There are shown in FIGS. 2A and 2B cross-section views of the surgical apparatus 100 being provided with the rotatable knob 40 having an internal key coupling for rotating the shaft assembly 30 relative to the handle assembly 20. In particular, the knob 40 includes a hub 92, at least one alignment hole 94, and at least one retaining pin 96 for coupling the knob 40 to the handle assembly 20 to allow 360° rotation of the shaft assembly 30 about an axis of the shaft assembly. The handle assembly 20 includes at least one circumferential groove 98 where the retaining pin 96 of the knob 40 can be placed, inserted or extended to allow 360° rotation. It is appreciated that rotation of the knob 40 and the shaft assembly 30 in relation to the handle assembly 20 requires at least one retaining pin 96 in the knob 40 extending into at least one circumferential groove 98 in the handle assembly 20. The knob 40 may further include a spring washer 93 (see FIG. 6C) between the distal end of the handle assembly 20 and the proximal end of the hub 92 to take up the linear play due to tolerance variations. The hub 92 may be formed of a plastic material and overmolded onto the outer tube 32 to provide secure coupling. The hub 92 may also be coupled to the outer tube 32 with an adhesive or other coupling means, such as a snap-fit or thermal welded connection. For example, the hub 92 may include detent tabs in the lumen and the outer tube 32 may include holes or other complimentary openings to accept the detent tabs for a mechanical coupling. This aspect can reduce the cost and time associated with overmolding, and simplify and facilitate assembly. FIG. 7 illustrates a proximal view of a rotatable knob 40a in accordance with another aspect of the invention having a plurality of ribs 99 providing a thin-walled knob while maintaining stability and reducing rotating friction with the handle assembly 20 and the shaft assembly 30, as well as permitting greater draft angles in the spaces between the ribs to facilitate removal from the mold during molding.

It is appreciated that the knob 40 may be formed in at least two pieces, for example, two identical halves that are joined together by interlocking or coupling means. The coupling means may include a snap-fit connection, an interference-fit connection, force-fit posts, or interconnecting tabs. With this aspect, the retaining pin(s) 96 would not be needed and may be replaced with at least one annular rib 99 in the lumen of the knob 40b as illustrated in FIG. 2C. The annular rib 99 would fit into the circumferential groove 98b on the handle assembly 20. In yet another aspect, the knob 40 may be molded as a single-piece component. With this aspect, the alignment hole(s) 94 and the retaining pin(s) 96 would not be needed and may be replaced with cantilever detent(s) that may be integrally molded with the knob 40. The cantilever detent(s) operate to snap into the circumferential groove(s) 98 of the handle assembly 20. Benefits of the above aspects include ease of assembly and smooth rotation of the knob.

In another aspect of the invention as illustrated in FIGS. 8A-8C, there are shown the fixed handle 22 and the pivoting handle 24 of the invention that can fit a variety of user's hand sizes. In particular, FIG. 8A illustrates the fixed handle 22 and the pivoting handle 24 without a snap-in ring installed for use with the largest size finger and thumb openings. FIG. 8B illustrates the fixed handle 22 with a small snap-in finger ring 22a, and the pivoting handle 24 with a medium snap-in thumb ring 24a. FIG. 8C illustrates the fixed handle 22 with the small snap-in finger ring 22a, and the pivoting handle 24 with a small snap-in thumb ring 24b. FIGS. 8A-8C illustrate the possibility of a variety of sizes of snap-in ring inserts to fit a user's hand size. It is appreciated that any possible combination of thumb and finger ring inserts can be included with each handle assembly 20 as typified in examples 20a, 20b and 20c of the surgical apparatus 100 so a user can select, for example, sizes between small and large. All the ring inserts may be formed of a softer material than the handle material to provide comfort to the user. In addition, the ring inserts can be made of different colors for identification of various sizes. It is appreciated that the handle assembly 20 of the surgical apparatus 100 can be used with or without the ring inserts.

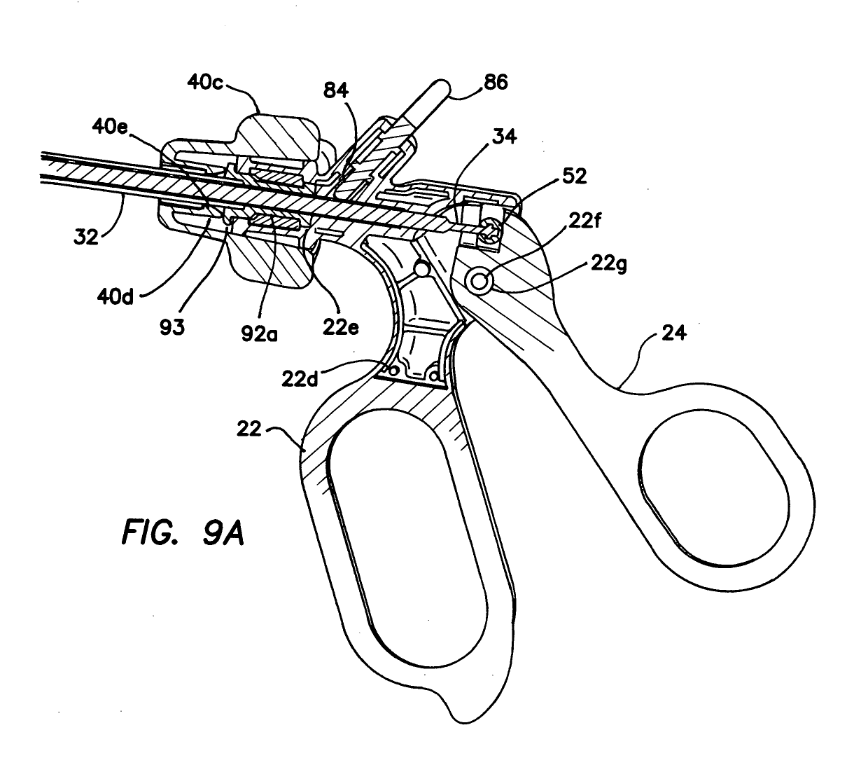

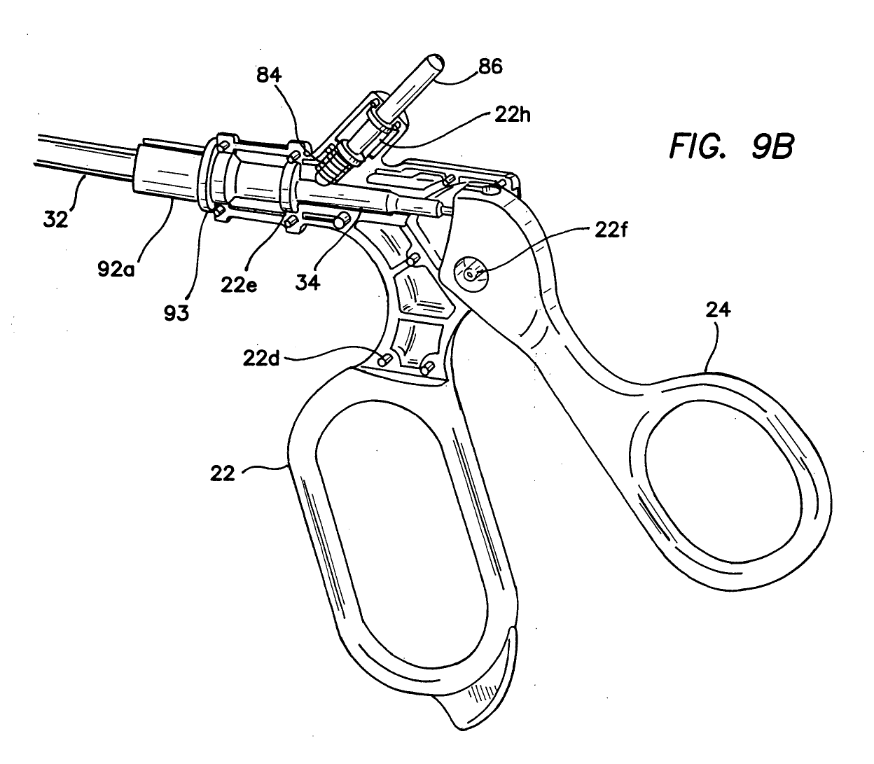

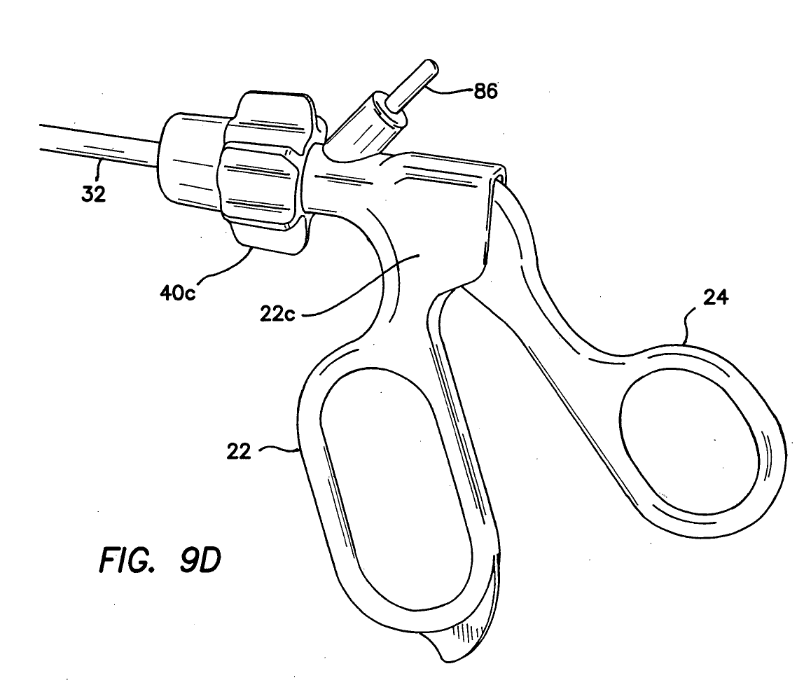

Referring to FIGS. 9A-9D, there is shown another aspect of the surgical instrument of the invention wherein the fixed handle 22 is formed of two pieces. In particular, the fixed handle 22 further includes a top cover 22c as illustrated in FIG. 9C, the top cover 22c being assembled onto the fixed handle 22 after the other components have been assembled or put in place, namely the pivoting handle 24, actuation rod 34, two-piece ball end 52, connector 86, spring 84, spring washer 93, and hub 92a. With this aspect, the top cover 22c may be fastened to the fixed handle 22 by means of press-fit posts 22d, or other means such as sonic welding, adhesive bonding, or other means well known in the art. The hub 92a may be formed of plastic and insert molded onto the outer tube 32, thus eliminating the need to cut the tube 32 to a precision length. The hub 92a rotates inside a groove 22e within the handle 22. A knob 40c, having cantilever detents 40d, slides onto the hub 92a and snaps into holes 40e of the hub 92a. Turning the knob 40c 360° rotates the hub 92a, which is coupled to the actuation rod 34 and the operative mechanism 210. A feature of this aspect of the invention is it facilitates assembly of the entire device. Moreover, the fixed handle 22 and the top cover 22c incorporate a press-fit post 22f and boss 22g to replace the press-fit pin 58 (FIG. 6C). In addition, the connector post 86 can freely rotate within the cavity 22h in the handle.

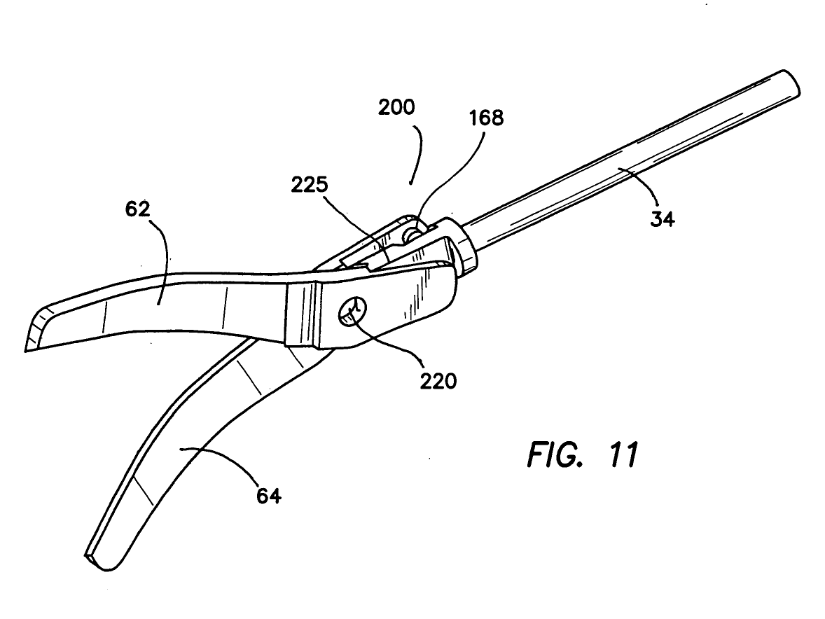

Referring to FIGS. 10A-10C, there is shown a surgical instrument 200 in accordance with an embodiment of the invention having an operative mechanism 210 including a first blade or tip 62 and a second blade or tip 64. As stated above, the first blade or tip 62 includes a first pin 158 protruding from the interfacing side 154 of the proximal-end portion 150 proximate a proximal end of the first tip and the second blade or tip 64 includes a second pin 168 (see FIG. 11) protruding from the interfacing side 164 of the proximal-end portion 160 proximate a proximal end of the second tip. The pins 158, 168 are fixed, such as by welding or other well known means, at the proximal ends of the blades or tips 62, 64. The blades or tips 62, 64 are overlapped in a scissors configuration and are held in a pivotal relationship by a common pin 220. The operative mechanism 210 interacts with a slotted actuation rod 34, as further explained below. It is appreciated that because the blades or tips 62, 64 include pins 158,168, rather than slots, the proximal-end portions 150, 160 of the blades or tips 62, 64 may include a smaller area than if the slots were positioned in the proximal-end portions of the blades or tips. This is beneficial because the “wingspan” of the proximal-end portions 150, 160 of the blades or tips 62, 64 when opened is minimized, if not eliminated.

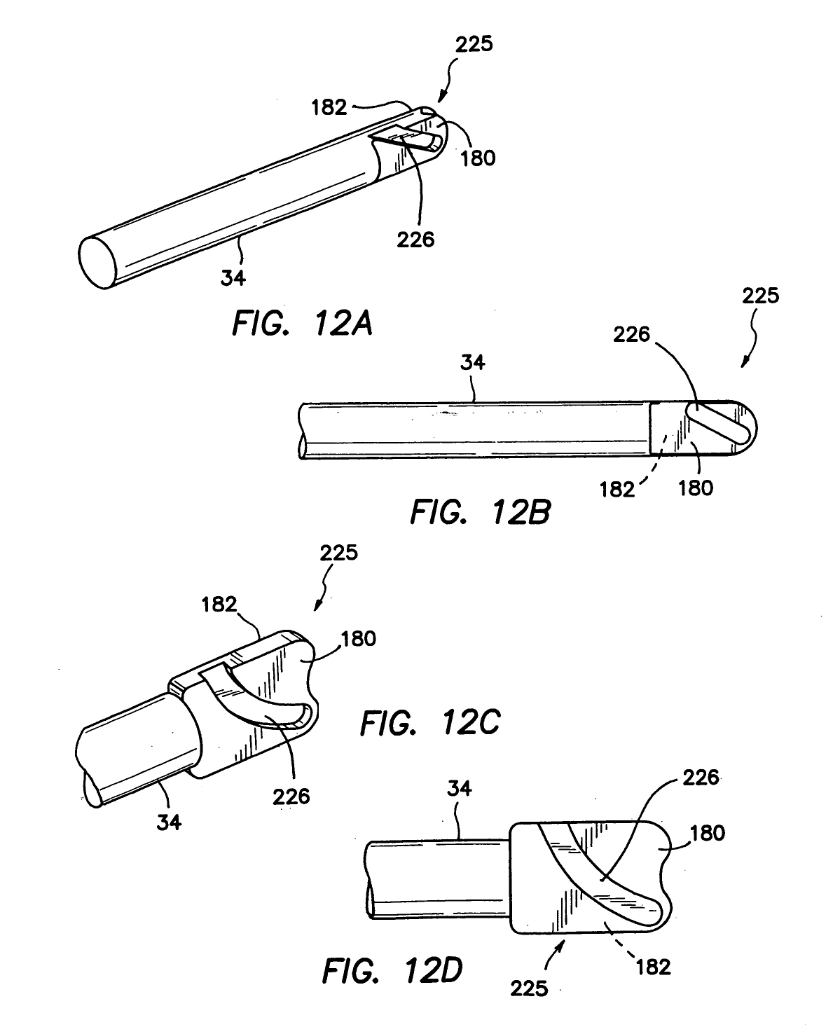

The actuation rod 34 includes a substantially flat tongue portion 225 at its distal-end region (see FIGS. 11 and 12A-12D). The tongue portion 225 includes a first substantially flat surface 180 and a second substantially flat surface 182 positioned opposite and substantially parallel to the first substantially flat surface. The tongue portion 225 may include a slot 226 on each of the first and second substantially flat surfaces 180, 182. Each slot corresponds to a pin 168, 158 on the first or second blade or tip 62, 64. The actuation rod 34 can be formed in a number of different ways. For example, the desired features can be machined from a solid rod or tube of a desired diameter. Alternatively, a strip of metal can be stamped with the desired slots within the tongue portion. Furthermore, the tongue portion, including the slots, may be machined or overmolded onto the actuation rod. The end of the actuation rod may also be formed as a separate part, i.e., molded, machined, cast, metal injection molded (MIM), etc., with the feature detail in it and then coupled to a standard length shaft by means of a thread, snap, adhesive, welding process or some other coupling means that is well known in the art.

Referring to FIG. 11, there is shown a perspective view of the operative mechanism 210 of FIGS. 10A-10C being coupled to the actuation rod 34. It is appreciated that there are numerous methods of manufacturing the blades or tips 62, 64. For example, the blades or tips 62, 64 can be formed from conventional stamping and then heat-treated. In another example, the blades or tips 62, 64 can be formed from a blank of pre-hardened material and then electro-discharge machine (EDM) cut, waterjet cut, laser cut or even machined to obtain the final shape. It should be noted that the pins 158, 168 in the proximal portions of the blades or tips 62, 64 can be formed directly onto the blades or they may be added in a later operation.

If the pins 158, 168 are not formed directly onto the blades or tips 62, 64, the pins may be coupled to the blades or tips in any one or a combination of ways, including press-fitted, swaged, threaded and/or welded. To manufacture the pin as part of the blades or tips 62, 64, a multitude of processes may be used. A sheet of material can be machined to make a blade or tip 62, 64 including a pivot hole and the pin. The sheet can then be heat treated and sent to a form grinder, which can grind one profile of the blade or tip. The ground plate can then be cut via EDM or other manufacturing method and the second profile can be cut out. This type of process can yield numerous components, with the drive pin integrally located, with relatively low cost.

There are other processes that can yield an entire blade or tip 62,64 from a minimum number of operations. These include, but are not limited to, MIM, casting, and power metallurgy (PM). Following one of these operations, the blade can be sharpened or receive other post-processing.

The pin and slot design of the invention provides a number of advantages. For example, the proximal portion of each blade or tip 62, 64 is reduced in area in comparison to prior art blades or tips so that during full deflection, very little or no part of the blade or tip extends beyond the inside diameter of the outer tube or shaft. This ensures that nothing catches on the blades or tips during use. This result may be attained because the area required for slots on the blades or tips is not needed. Additionally, by moving the drive slots 226 to the actuation rod 34, the usable area for the drive slots is increased. Further, with the slots positioned on the actuation rod, the depth of the slots can be varied in order to increase tension on the blades during actuation. Another advantage is that by moving the slot from the blades or tips to the actuation rod, the “wingspan” of the blades can be reduced or eliminated because the proximal portion of the blades or tips does not need to encase the slot. Instead, the proximal portion of the blades or tips include only a small pin, which minimizes the chance of the blades or tips catching on tissue, other instruments or sutures.

Referring to FIGS. 12A and 12B, there is shown a perspective view and a side view of the actuation rod 34 incorporating slots 226 on both sides of the tongue end, respectively. As explained above, the blades or tips can have pins at the proximal portions of the blades or tips that nest in the slots of the rod. The rod may be pushed forward or pulled backwards to cam the blades or tips, which are pivoted at a common pivot point and coupled to the insert 70, 70a, and 70b. In some cases it may be beneficial to have different slot designs to actuate the tips to different opening angles or distances, at different speeds, for different length tips and for varying force. The slots 226 may include an open or closed end slot (or combination of both) as desired. FIGS. 12C and 12D illustrate an actuation rod having a curved slot in accordance with another aspect of the invention. A curved slot may be used to provide a more linear relation between the actuation rod and the jaw motion. For example, the slot can be shaped to provide more control as the blades or tips are nearing the closed position, and greater acceleration as the blades or tips are near the opened position. With this aspect, the instrument can be tuned to provide the desired control and user feedback.

It will be understood that many other modifications can be made to the various disclosed embodiments without departing from the spirit and scope of the invention. For these reasons, the above description should not be construed as limiting the invention, but should be interpreted as merely exemplary of preferred embodiments. It will be understood that many other modifications can be made to the various disclosed embodiments without departing from the spirit and scope of the invention. For example, various sizes of the surgical device are contemplated as well as various types of constructions and materials. It will also be apparent that many modifications can be made to the configuration of parts as well as their interaction. For these reasons, the above description should not be construed as limiting the invention, but should be interpreted as merely exemplary of embodiments.

1. A surgical instrument, comprising:
a single elongate tube extending along an axis, the elongate tube including a proximal end and a distal end, the elongate tube being made of an electrically nonconductive material;
an actuation rod including a proximal end and a distal end, the actuation rod being coaxially slidable within the elongate tube, the proximal end of the actuation rod extending proximally from the proximal end of the elongate tube, the distal end of the actuation rod being positioned within a distal-end region of the elongate tube, a distal-end region of the actuation rod including a tongue portion, the tongue portion including a first substantially flat surface and a second substantially flat surface positioned opposite and substantially parallel to the first substantially flat surface, the first flat surface of the tongue including a first inclined slot, the second flat surface of the tongue including a second inclined slot transverse to the first slot, the actuation rod providing flexural strength to the surgical instrument;
a first tip including a proximal-end portion, a distal-end portion, an interfacing side and an opposed side, with a first pin protruding from the interfacing side of the proximal-end portion proximate a proximal end of the first tip;
a second tip including a proximal-end portion, a distal-end portion, an interfacing side and an opposed side, with a second pin protruding from the interfacing side of the proximal-end portion proximate a proximal end of the second tip; and
a pivot pin pivotally coupling the second tip to the first tip, the pivot pin being operably coupled to the elongate tube to open and close the tips in response to movement of the actuation rod,
wherein the first inclined slot of the tongue portion of the actuation rod accepts the first pin of the first tip and the second inclined slot of the tongue portion of the actuation rod accepts the second pin of the second tip,
the interfacing surface of the first tip and the interfacing surface of the second tip interface with each other,
the first inclined slot including camming surfaces for the first pin to slide along within the first inclined slot, and
the second inclined slot including camming surfaces for the second pin to slide along within the second inclined slot.

2. The surgical instrument of claim 1, wherein at least one of the first inclined slot and the second inclined slot is curved.

3. The surgical instrument of claim 1, wherein at least one of the first inclined slot and the second inclined slot is an open-end slot.

4. The surgical instrument of claim 1, wherein at least one of the first inclined slot and the second inclined slot is a closed-end slot.

5. The surgical instrument of claim 1, the actuation rod comprising an electrically conductive material.

6. A surgical instrument, comprising:
a handle assembly including a fixed handle and a pivoting handle;
a shaft assembly extending distally from the handle assembly, the shaft assembly including:
a single elongate tube extending along an axis, the elongate tube including a proximal end and a distal end, the elongate tube being made of an electrically nonconductive material;
an actuation rod including a proximal end and a distal end, the actuation rod being coaxially slidable within the elongate tube, the proximal end of the actuation rod extending proximally from the proximal end of the elongate tube, the distal end of the actuation rod being positioned within a distal-end region of the elongate tube, the proximal end of the actuation rod including a ball end that couples to the pivoting handle, a distal-end region of the actuation rod including a tongue portion, the tongue portion including a first substantially flat surface and a second substantially flat surface positioned opposite and substantially parallel to the first substantially flat surface, the first flat surface of the tongue including a first inclined slot, the second flat surface of the tongue including a second inclined slot transverse to the first slot, the actuation rod providing flexural strength to the surgical instrument; and
a knob adapted to provide 360° rotation of the sleeve assembly about the axis of the elongate tube relative to the handle assembly, the knob being coupled to the exterior surface of the elongate tube proximate the proximal end of the elongate tube and coupled to the handle assembly;
an operative mechanism including a first tip and a second tip, the first tip including a proximal-end portion, a distal-end portion, an interfacing side and an opposed side, with a first pin protruding from the interfacing side of the proximal-end portion proximate a proximal end of the first tip, the second tip including a proximal-end portion, a distal-end portion, an interfacing side and an opposed side, with a second pin protruding from the interfacing side of the proximal-end portion proximate a proximal end of the second tip, the interfacing side of the first tip and the interfacing side of the second tip interfacing with each other; and
a pivot pin pivotally coupling the second tip to the first tip, the pivot pin being operably coupled to the distal-end region of the elongate tube to open and close the tips in response to movement of the actuation rod,
wherein the first inclined slot of the tongue portion of the actuation rod accepts the first pin of the first tip and the second inclined slot of the tongue portion of the actuation rod accepts the second pin of the second tip,
the first inclined slot including camming surfaces for the first pin to slide along within the first inclined slot, and
the second inclined slot including camming surfaces for the second pin to slide along within the second inclined slot.

7. The surgical instrument of claim 6, the pivoting handle including a groove and the ball end of the actuation rod fitting into the groove to form a rotatable ball-and-socket joint.

8. The surgical instrument of claim 7, wherein the fixed handle encloses the pivoting handle.

9. The surgical instrument of claim 6, the operative mechanism including a pair of scissors.

10. The surgical instrument of claim 6, the operative mechanism including a clamp.

11. The surgical instrument of claim 6, the shaft assembly further comprising a substantially cylindrical insert having a proximal end and a distal end with a lumen therebetween, the insert including an aperture substantially perpendicular to an axis of the insert for accepting the pivot pin, the insert being coupled to the inner surface of the elongate tube proximate the distal end of the elongate tube, at least a portion of the proximal-end portion of the first tip and the proximal-end portion of the second tip being positioned within the lumen of the insert.

12. The surgical instrument of claim 11, wherein:
the pivot pin includes a screw, and
the aperture in the insert includes threads for receiving the threads of the pivot pin.

13. The surgical instrument of claim 11, wherein the insert is press fit into the elongate tube.

14. The surgical instrument of claim 11, wherein the insert is coupled to the elongate tube via an adhesive.

15. The surgical instrument of claim 11, wherein the insert is formed of a plastic material.

16. The surgical instrument of claim 11, wherein the insert is formed of a metallic material.

17. The surgical instrument of claim 11, wherein the insert includes a proximal clevis extending longitudinally and a distal clevis extending longitudinally, the proximal clevis and the distal clevis being substantially rotatably aligned with each other about the axis of the insert, the proximal clevis and the distal clevis being substantially parallel to the axis of the insert, and the proximal clevis and the distal clevis being substantially perpendicular to the aperture of the insert, the proximal clevis sandwiching the proximal-end portions of the first tip and the second tip between the tongue portion of the actuation rod and the walls of the proximal clevis.

18. The surgical instrument of claim 6, wherein at least one of the first inclined slot and the second inclined slot is curved.

19. The surgical instrument of claim 6, wherein at least one of the first inclined slot and the second inclined slot is an open-end slot.

20. The surgical instrument of claim 6, wherein at least one of the first inclined slot and the second inclined slot is a closed-end slot.

21. A surgical instrument, comprising:
a handle assembly including a fixed handle and a pivoting handle, the pivoting handle including a groove;
a shaft assembly extending distally from the handle assembly, the shaft assembly including:
a single elongate tube extending along an axis, the elongate tube including a proximal end and a distal end, the elongate tube being made of an electrically nonconductive material;
an actuation rod including a proximal end and a distal end, the actuation rod being coaxially slidable within the elongate tube, the proximal end of the actuation rod extending proximally from the proximal end of the elongate tube, the distal end of the actuation rod being positioned within a distal-end region of the elongate tube, the proximal end of the actuation rod including a ball end that couples to the groove of the pivoting handle to form a rotatable ball-and-socket joint, a distal-end region of the actuation rod including a tongue portion, the tongue portion including a first substantially flat surface and a second substantially flat surface positioned opposite and substantially parallel to the first substantially flat surface, the first flat surface of the tongue including a first inclined slot, the second flat surface of the tongue including a second inclined slot transverse to the first slot, the actuation rod providing flexural strength to the surgical instrument;
a substantially cylindrical insert having a proximal end and a distal end with a lumen therebetween, the insert including an aperture substantially perpendicular to an axis of the insert, the insert being coupled to the inner surface of the elongate tube proximate the distal end of the elongate tube, the insert including a proximal clevis extending longitudinally and a distal clevis extending longitudinally, the proximal clevis and the distal clevis being substantially rotatably aligned with each other about the axis of the insert, the proximal clevis and the distal clevis being substantially parallel to the axis of the insert, and the proximal clevis and the distal clevis being substantially perpendicular to the aperture of the insert; and
a knob adapted to provide 360° rotation of the sleeve assembly about the axis of the elongate tube relative to the handle assembly, the knob being coupled to the exterior surface of the elongate tube proximate the proximal end of the elongate tube and coupled to the handle assembly;
an operative mechanism including a first tip and a second tip, the first tip including a proximal-end portion, a distal-end portion, an interfacing side and an opposed side, with a first pin protruding from the interfacing side of the proximal-end portion proximate a proximal end of the first tip, the second tip including a proximal-end portion, a distal-end portion, an interfacing side and an opposed side, with a second pin protruding from the interfacing side of the proximal-end portion proximate a proximal end of the second tip, the interfacing side of the first tip and the interfacing side of the second tip interfacing with each other; and
a pivot pin pivotally coupling the second tip to the first tip, the pivot pin being operably coupled to the distal-end region of the elongate tube to open and close the tips in response to movement of the actuation rod,
wherein the first inclined slot of the tongue portion of the actuation rod accepts the first pin of the first tip and the second inclined slot of the tongue portion of the actuation rod accepts the second pin of the second tip,
the first inclined slot including camming surfaces for the first pin to slide along within the first inclined slot,
the second inclined slot including camming surfaces for the second pin to slide along within the second inclined slot,
the aperture of the insert being adapted for accepting the pivot pin;
at least a portion of the proximal-end portion of the first tip and the proximal-end portion of the second tip being positioned within the lumen of the insert, and
the proximal clevis of the insert sandwiching the proximal-end portions of the first tip and the second tip between the tongue portion of the actuation rod and the walls of the proximal clevis of the insert.

22. The surgical instrument of claim 21, the operative mechanism including a pair of scissors.

23. The surgical instrument of claim 21, the operative mechanism including a clamp.

24. The surgical instrument of claim 21, wherein the fixed handle encloses the pivoting handle.

25. The surgical instrument of claim 21, wherein:
the pivot pin includes a screw, and
the aperture in the insert includes threads for receiving the threads of the pivot pin.

26. The surgical instrument of claim 21, wherein the insert is formed of a plastic material.

27. The surgical instrument of claim 21, wherein the insert is formed of a metallic material.

28. The surgical instrument of claim 21, the knob being formed in at least two pieces.

29. The surgical instrument of claim 28, the at least two pieces of the knob being joined together by a coupling means.

30. The surgical instrument of claim 28, wherein:
the handle assembly further including a circumferential groove at a distal portion of the handle assembly, and
the knob including at least one annular rib in a lumen of the knob, the at least one annular rib fitting into the circumferential groove on the handle assembly.

31. The surgical instrument of claim 21, the knob being formed as a single-piece component, a lumen of the knob including at least one cantilever detent.

32. The surgical instrument of claim 31, wherein:
the handle assembly further including a circumferential groove at a distal portion of the handle assembly, and
the at least one cantilever detent engaging the circumferential grove of the handle assembly.
